(19) United States
(12) Patent Application Publication
(10) Pub. No.: US 2012/0195875 A1
(43) Pub. Date: Aug. 2, 2012

(54) DETECTION OF IMMUNE MODULATION RESULTING FROM REDUCED PROTEIN PHOSPHATASE 2A ACTIVITY

(75) Inventors: Ozlem Equils, Sherman Oaks, CA (US); Kathrin Michelsen, Los Angeles, CA (US)

(73) Assignee: CEDARS-SINAI MEDICAL CENTER, Los Angeles, CA (US)

(21) Appl. No.: 13/360,439

(22) Filed: Jan. 27, 2012

Related U.S. Application Data
(60) Provisional application No. 61/436,859, filed on Jan. 27, 2011.

(51) Int. Cl. A61K 38/46; A61P 31/00; A61P 29/00; A61P 31/10; A61P 31/12; A61P 1/00; C12Q 1/42; A61P 31/04

(52) U.S. Cl. 424/94.6; 435/21

(57) ABSTRACT

The inventors demonstrate herein that measuring the level of protein phosphatase 2A activity (PP2A) is useful for assessing immune modulation and susceptibility to infection in an individual. This invention is especially useful when applied to septic individuals, and individuals with chronic infections. The invention also teaches a method of prevention and treatment of secondary infections, as well as prevention and treatment of cancerous conditions.

### CROSS-REFERENCE TO RELATED APPLICATIONS

This application claims priority from U.S. Provisional Patent Application No. 61/436,859 filed on Jan. 27, 2011, which is incorporated herein by reference in its entirety.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. AI51216 awarded by the National Institutes of Health.

### FIELD OF INVENTION

The present invention relates to assessing immune modulation and susceptibility to infection in individuals, including those with sepsis and chronic infections. The present invention further relates to the identification of individuals who are at risk for cancer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The detection of pathogens by innate immune cells triggers a robust and essential inflammatory reaction. However, uncontrolled inflammation leads to excessive tissue destruction and manifestation of pathological states like sepsis, autoimmune diseases, and cancer, making a tight control of the inflammatory reaction necessary. One important mechanism to prevent over-exuberant inflammation is the induction of endotoxin tolerance. On the other hand prolonged and severe immune tolerance can increase the risk for secondary bacterial infections. Despite numerous studies the molecular mechanisms underlying LPS tolerance in vitro and in vivo remain largely elusive (12). In particular, the effect of LPS-induced tolerance on protein phosphatase activity remains largely unknown.

Currently there is no diagnostic test available to assess the level of immune suppression in septic patients, and there is no treatment available to reverse the immune modulation. Further, although infection has been shown to be associated with cancer development, there are no effective prevention strategies. Therefore, there is a need in the art to identify a diagnostic marker to assess the level of immune modulation in septic patients, and in patients with chronic infections, to provide a method of prevention and treatment of secondary infections, as well as prevention and treatment of cancerous conditions.

### SUMMARY OF THE INVENTION

In some embodiments, the invention teaches a method of determining an individual's susceptibility to an infection, including: obtaining a biological sample from the individual; measuring a level of protein phosphatase 2A (PP2A) activity in the biological sample; and determining the individual's susceptibility to the infection based upon the level of PP2A activity, wherein a higher than average risk of developing the infection is determined if abnormally low PP2A activity is measured, and an average risk of developing the infection is determined if normal PP2A activity is measured. In some embodiments, the level of PP2A activity is measured indirectly by measuring the level of phosphorylation of its upstream and/or downstream interacting/target molecules in the biological sample. In some embodiments, the individual is immune suppressed. In some embodiments, the individual has sepsis. In some embodiments, the infection is a superinfection. In some embodiments, the infection is selected from the group consisting of: bacterial, fungal, viral, and combinations thereof. In some embodiments, the individual has a history of infection. In some embodiments, the infection is a gram-negative and/or gram-positive bacterial infection.

In some embodiments, the invention teaches a method of reducing a likelihood of an individual developing an infection, including: providing a prophylactic dose of protein phosphatase 2A (PP2A), and administering the dose to the individual. In some embodiments, the individual is immune suppressed. In some embodiments, the infection is a superinfection. In some embodiments, the infection is selected from the group consisting of: bacterial, fungal, viral, and combinations thereof. In some embodiments, the infection is a gram-negative and/or gram-positive bacterial infection. In some embodiments, the individual has sepsis and the infection is a secondary infection.

In some embodiments, the invention teaches a method of treating an infection in an individual, including: providing a therapeutic dose of protein phosphatase 2A (PP2A), and administering the dose to the individual. In some embodiments, the individual is immune suppressed. In some embodiments, the infection is a superinfection. In some embodiments, the infection is selected from the group consisting of: bacterial, fungal, viral, and combinations thereof. In some embodiments, the infection is a gram-negative and/or gram-positive infection.

In certain embodiments, the invention teaches a kit for determining an individual's susceptibility to infection, including: a means for measuring a level of protein phosphatase 2A (PP2A) activity in a biological sample of the individual, and instructions for use thereof to determine an individual's susceptibility to infection. In some embodiments, the level of PP2A activity is measured indirectly by measuring the level of phosphorylation of its upstream and/or downstream interacting/target molecules in the biological sample. In some embodiments, the infection is selected from the group consisting of: viral, bacterial, fungal, and combinations thereof.

In certain embodiments, the invention teaches a kit for treating an infection in an individual, including: a quantity of protein phosphatase 2A (PP2A), and instructions for use thereof to treat an infection in an individual. In some embodiments, the infection is selected from the group consisting of: viral, bacterial, fungal, and combinations thereof.

In certain embodiments, the invention teaches a kit for reducing the likelihood of an individual contracting an infection, including: a quantity of protein phosphatase 2A (PP2A), and instructions for use thereof for reducing the likelihood of an individual contracting an infection. In some embodiments, the infection is selected from the group consisting of: viral, bacterial, fungal, and combinations thereof.

In certain embodiments, the invention teaches a method of determining an individual's susceptibility to cancer, including: obtaining a biological sample from the individual; measuring a level of protein phosphatase 2A (PP2A) activity in the biological sample; and determining the individual's susceptibility to cancer based upon the level of PP2A activity, wherein a higher than average risk of developing cancer is determined if abnormally low PP2A activity is measured, and an average risk of developing cancer is determined if normal PP2A activity is measured. In some embodiments, the level of PP2A activity is measured indirectly by measuring the level of phosphorylation of its upstream and/or downstream interacting/target molecules in the biological sample. In some embodiments, the cancer is the result of repeated infections and/or reduced PP2A activity.

In some embodiments, the invention teaches a kit for determining an individual's susceptibility to cancer, including: a means for measuring a level of protein phosphatase 2A (PP2A) activity in a biological sample of the individual, and instructions for use thereof to determine an individual's susceptibility to cancer. In some embodiments, the level of PP2A activity is measured indirectly by measuring the level of phosphorylation of its upstream and/or downstream interacting/target molecules in the biological sample. In some embodiments, the cancer is the result of repeated infections and/or reduced PP2A activity.

In some embodiments, the invention teaches a method of treating an individual after determining the individual has a higher than average risk of developing an infection resulting from abnormally low PP2A activity, including: obtaining a biological sample from the individual; measuring a level of protein phosphatase 2A (PP2A) activity in the biological sample; determining the individual's susceptibility to the infection based upon the level of PP2A activity, wherein a higher than average risk of developing the infection is determined if abnormally low PP2A activity is measured, and an average risk of developing the infection is determined if normal PP2A activity is measured; and treating the individual with a prophylactic dose of a type of medication selected from the group consisting of: antiviral, antifungal, antibacterial, and combinations thereof if it is determined the individual has a higher than average risk of developing the infection resulting from abnormally low PP2A activity. In some embodiments, the level of PP2A activity is measured indirectly by measuring the level of phosphorylation of its upstream and/or downstream interacting/target molecules in the biological sample.

In certain embodiments, the invention teaches a method of monitoring an individual after determining the individual has a higher than average risk of developing cancer resulting from abnormally low PP2A activity, including: obtaining a biological sample from the individual; measuring a level of protein phosphatase 2A (PP2A) activity in the biological sample; determining the individual's susceptibility to cancer based upon the level of PP2A activity measured, wherein a higher than average risk of developing cancer is determined if abnormally low PP2A activity is measured, and an average risk of developing cancer is determined if normal PP2A activity is measured; and testing the individual for one or more forms of cancer more frequently than an individual having an average risk of developing cancer if it is determined the individual has a higher than average risk of developing cancer resulting from abnormally low PP2A activity. In some embodiments, the level of PP2A activity is measured indirectly by measuring the level of phosphorylation of its upstream and/or downstream interacting/target molecules in the biological sample.

In certain embodiments, the invention teaches a method of treating an individual after determining the individual has a higher than average risk of developing cancer of the digestive tract resulting from abnormally low PP2A activity, including: obtaining a biological sample from the individual; measuring a level of protein phosphatase 2A (PP2A) activity in the biological sample; determining the individual's susceptibility to cancer of the digestive tract based upon the level of PP2A activity, wherein a higher than average risk of developing cancer of the digestive tract is determined if abnormally low PP2A activity is measured, and an average risk of developing cancer of the digestive tract is determined if normal PP2A activity is measured; and treating the individual with antibiotics if it is determined the individual has a higher than average risk of developing cancer of the digestive tract resulting from abnormally low PP2A activity. In some embodiments, the individual has been diagnosed with inflammatory bowel disease. In some embodiments, the level of PP2A activity is measured indirectly by measuring the level of phosphorylation of its upstream and/or downstream interacting/target molecules in the biological sample.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 demonstrates, in accordance with an embodiment of the invention, LPS-tolerization persistently suppresses PP2A activity in THP-1 cells. THP-1 cells were treated with media or LPS (10 ng/ml) for various durations of time. The cells were washed and treated again with LPS (100 ng/ml) for 5 hours and PP2A activity was assessed using a colorimetric PP2A assay kit (Upstate). Maximum PP2A activity suppression was observed at 24 h of primary LPS stimulation. This suppression was detectable for 48 h of primary LPS stimulation. One representative experiment out of three independent experiments with similar results is shown. Means±SD are shown (* p<0.05 LPS/LPS compared to M/LPS treated cells).

FIG. 2 demonstrates, in accordance with an embodiment of the invention, ex vivo tolerization of mouse peritoneal macrophages leads to decreased LPS-induced IL-6 and TNF-α release. Mouse peritoneal macrophages were isolated from C57BL/6 mice. The cells were then treated with LPS (10 ng/ml) overnight followed by a second dose of LPS (100 ng/ml) for 5 hours, IL-6 (A) or TNF-α (B) release were measured by ELISA 5 hours after the second LPS treatment. The inventors observed that repeated LPS treatment leads to the tolerization of mouse peritoneal macrophages in vitro as assessed by suppressed IL-6 or TNF-α release. One representative experiment out of three independent experiments with similar results is shown. Means±SD of quadruplicate stimulations are shown (* p<0.05 LPS/LPS compared to M/LPS treated cells).

FIG. 3 demonstrates, in accordance with an embodiment of the invention, in vivo tolerization of mice leads to decreased LPS-induced TNF-α and IL-6 release. Female C57BL/6 mice were treated with either PBS or LPS i.p. (25 μg/mouse) overnight. The inventors then treated the animals with either PBS or LPS i.p. (25 μg/mouse). The blood was collected at the indicated time-points and IL-6 (A) or TNF-α (B) serum concentrations were measured by ELISA. The inventors observed that repeated LPS treatment led to tolerization and suppressed TNF-α and IL-6 release in mice in vivo. One representative experiment out of three experiments with similar results is shown. Means are shown (* p<0.05 LPS/LPS compared to PBS/LPS treated cells, n=3 mice per group).

FIG. 4 demonstrates, in accordance with an embodiment of the invention, PP2A activity is reduced in in vivo tolerized mice. Female C57BL/6 mice were tolerized to LPS as described in FIG. 4. Peritoneal macrophages were isolated from tolerized or non-tolerized mice and PP2A activity was measured. One representative experiment out of two experiments with similar results is shown. Means are shown (* p<0.05 LPS/LPS compared to PBS/LPS or PBS/PBS treated cells, n=3 mice per group).

FIG. 5 demonstrates, in accordance with an embodiment of the invention, exogenous PP2A restores the LPS-induced TNF response in tolerized THP-1 cells. THP-1 cells were treated with media or LPS (10 ng/ml) overnight, washed and preincubated with PP2A for 1 h followed by stimulation with LPS (100 ng/ml) for 5 h. Supernatants were harvested and assessed for TNF-α release by ELISA. Repeated exposure to LPS lead to tolerization of THP-1 cells as measured by reduced TNF-α secretion. The inventors observed that PP2A treatment prior to the 1^{st} LPS treatment had no effect on TNF-α release from the THP-1 cells. In contrast, PP2A treatment prior to the second LPS stimulation restored the LPS responsiveness at 5 hours. One representative experiment out of 2 independent experiments with similar results is shown. Means±SD are shown (* p<0.05 LPS/LPS compared to M/LPS treated cells).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein:

The acronym “LPS” means lipopolysaccharide.

The acronym “PBMC” means peripheral blood mononuclear cell.

The acronym “MDM” means monocyte-derived macrophages.

The acronym “SOCS” means suppressor of cytokine signaling.

The acronym “PP2A” means protein phosphatase 2A.

As used herein, “beneficial results” may include, but are in no way limited to, lessening or alleviating the severity of the disease condition, preventing the disease condition from worsening, curing the disease condition, preventing the disease condition from developing, lowering the chances of a subject developing the disease condition and prolonging a subject's life or life expectancy.

“Conditions” and “disease conditions,” as used herein may include, but are in no way limited to any form of acute infection, chronic infection, sepsis, super-infection, immune suppression, cancerous conditions, or combinations thereof.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

By way of background, macrophages exposed to enteric gram-negative lipopolysaccharide (LPS) become refractory to subsequent challenge with LPS. This refractory phenotype, referred to as LPS-tolerance, is characterized by a decline in the production of inflammatory cytokines, including tumor necrosis factor (TNF)-α, interleukin (IL)-6, and IL-12 (1-3). LPS-tolerance was first demonstrated in animal models of endotoxemia (4), and this phenotype can also be induced in primary monocytes, monocyte-derived macrophages (MDM), and mature macrophages as well as in myeloid cell lines (5-8). In humans, this condition is observed in peripheral blood mononuclear cells (PBMC) isolated from subjects given a single intravenous dose of Escherichia coli LPS (9) and in monocytes isolated from patients with sepsis (10,11) where it contributes to “immunosuppression”, secondary infections, and mortality.

Although the molecular mechanisms underlying tolerance to microbial products have been studied extensively they are still largely unknown (12-14). Interaction of LPS with its cellular receptor TLR4 triggers a multitude of signaling events including the phosphorylation of MAPKs and IκB leading to the activation of NF-κB responsive proinflammatory cytokines such as TNF-α. (15,16). The activation of MAPKs and NF-κB are tightly regulated and dysregulation of these signaling pathways have been implemented in LPS tolerance. Decreased expression of the LPS receptor complex TLR4/MD2 (17), suppressed IRAK-1-MyD88 interactions and IRAK-1 activation (14, 18-22), as well as decreased TLR4 tyrosine phosphorylation and consequently poor MyD88 recruitment to TLR4 (23) were suggested to mediate LPS tolerance. Others have shown an increase in negative regulators of TLR4 signaling, such as Toll-interacting protein (Tollip), suppressor of cytokine signaling (SOCS)-1, IL-1R-associated kinase-M (IRAK-M), ST2, an alternatively spliced short version of MyD88 (MyD88s), and SH2-containing inositol phosphatase (SHIP-1) expression in tolerized cells (24-28).

Following initiation of the above mentioned signaling pathways de-phosphorylation plays a central role in regulating and limiting intracellular signaling. Since phosphorylation of several signaling molecules, such as IRAK-1, MAPKs, PKR, and IKB is impaired in LPS tolerant cells (23,29) the inventors explored the possible role of phosphatases in LPS-induced tolerance in macrophages. Furthermore, SH2-containing inositol phosphatase (SHIP-1) has been implicated in endotoxin tolerance (28) but the involvement of other phosphatases has not been studied.

The serine-threonine protein phosphatase PP2A is abundantly expressed in cells and has been implicated in the regulation of many signaling pathways including those implicated in endotoxin tolerance such as JNK, and MAPK pathways (30,31). Inhibition of PP1 and PP2A by calyculin A has been shown to inhibit LPS-induced TNF-α, IL-1β, IFN-β, IP-10, IRF-1, and TNFR-2 release in macrophages (32). In addition to tyrosine kinases, serine-threonine kinases have been proposed to regulate LPS-induced signaling in macrophages (27). Briefly, PP2A associates with IKK and leads to the modulation of NF-KB transcriptional activity (33,34) by directly interacting with IKKβ (34).

Toll-like receptors are sensors for microbial components leading to the secretion of pro-inflammatory mediators by monocytes/macrophages. This process is tightly regulated by various mechanisms. Repetitive exposure of macrophages to bacterial lipopolysaccharide (LPS) is known to tolerize them to further stimulation and inhibit pro-inflammatory cytokine release.

The inventors demonstrate herein that PP2A activity is suppressed in vitro in lipopolysaccharide (LPS)-tolerized human THP-1 macrophages, and in vivo in peritoneal macrophages obtained from mice repeatedly exposed to LPS. These observations have important clinical implications in understanding the mechanisms associated with tolerization and consequences of repeated LPS exposure.

In certain embodiments, the present invention teaches a method of determining an individual's susceptibility to infection by obtaining a biological sample from the individual and determining a level of PP2A activity in the biological sample. In certain embodiments, a higher than average risk of developing an infection is determined if abnormally low PP2A activity is measured, and an average risk of developing an infection is determined if normal PP2A activity is measured. In some embodiments, PP2A activity is determined indirectly by measuring the level of phosphorylation of its upstream and/or downstream interacting/target molecules in the biological sample. In certain embodiments, the susceptibility determined is susceptibility to a bacterial, fungal or viral infection, or combinations thereof. In some embodiments, the susceptibility determined is susceptibility to a superinfection. In certain embodiments, the susceptibility determined is susceptibility to a gram-negative and/or gram-positive bacterial infection. In various embodiments, the individual tested for susceptibility has a history of a gram-negative and/or gram-positive bacterial infection, fungal infection, or viral infection. In certain embodiments, the individual is a mammal. In certain embodiments, the individual is a human.

One of skill in the art would readily appreciate there are many ways to measure PP2A activity. In certain embodiments, PP2A activity is measured according to the protocols set forth in Examples 6 and 10, disclosed herein. In certain embodiments, PP2A activity is measured utilizing an immunologically-based assay to analyze the biological sample. In certain embodiments, PP2A activity is measured using a spectroscopy-based assay to analyze the biological sample. In certain embodiments PP2A activity may be measured alone. In other embodiments, PP2A activity may be measured in combination with other phosphatases. In certain embodiments, PP2A activity may be measured indirectly by measuring the level of phosphorylation of its upstream and/or downstream interacting/target molecules.

In certain embodiments, the present invention teaches a method of reducing an individual's likelihood of developing an infection by administering to the individual a prophylactic dose of PP2A. In some embodiments, the individual is immune suppressed. In some embodiments, the infection is a superinfection. In some embodiments, the PP2A is administered in combination with prophylactic doses of other phosphatases and/or other therapeutic agents. In certain embodiments, the PP2A administered is recombinant PP2A. In yet other embodiments, a substitute agent for PP2A with substantially similar physical properties and/or physiological effect with respect to the ability of PP2A to reduce an individual's likelihood of developing an infection is administered to the individual. In certain embodiments, the source of the immune suppression in the individual is prior exposure to LPS from a gram-negative bacterial infection or antigens from gram-positive bacterial infections, fungal, or viral infections. In certain embodiments, the individual is a mammal. In certain embodiments, the individual is a human.

In certain embodiments, the present invention teaches a method of treating an infection in an individual by administering to the individual a therapeutic dose of PP2A. In certain embodiments, the individual is immune suppressed. In some embodiments, improved immune function results from treatment. In certain embodiments, the PP2A administered is recombinant PP2A. In yet other embodiments, a substitute agent for PP2A with substantially similar physical properties and/or physiological effect with respect to the ability of PP2A to treat an infection in an individual is administered to the individual. In some embodiments, endogenous expression of PP2A is upregulated in order to treat, prevent or reduce the likelihood of an infection. Upregulation of PP2A can be achieved by any number of methods, as will be readily appreciated by one of skill in the art. For example, a vector may be used to introduce additional copies of PP2A, in order to increase expression thereof. In some embodiments, the vector is an adenovirus. One of skill in the art would readily appreciate that any number of other viruses with substantially similar capacities for expression could be used. Alternatively, endogenous expression of PP2A may be upregulated by treating the individual with one or more substances that naturally induce greater expression of PP2A, either directly, or indirectly. In certain embodiments, the infection is a superinfection. In some embodiments, the infection is caused by gram-negative bacteria, gram-positive bacteria, fungi, virus, or combinations thereof. In some embodiments, the administration of PP2A is part of a treatment protocol that includes one or more additional therapeutic agents.

In certain embodiments, the present invention teaches a method of determining an individual's susceptibility to cancer secondary to chronic infection by obtaining a biological sample from the individual, measuring a level of PP2A activity in the sample, and determining the individual's risk of developing cancer, based upon the level of PP2A activity, alone or in combination with other phosphatases, with or without the presence of other risk factors and with or without other laboratory test results. In certain embodiments, a higher than average risk of developing cancer is determined if abnormally low PP2A activity is measured, alone or in combination with other phosphatases, and an average risk of developing cancer is determined if normal PP2A activity is measured, alone or in combination with other phosphatases. In some embodiments, PP2A activity is determined indirectly by measuring the level of phosphorylation of its upstream and/or downstream interacting/target molecules in the biological sample.

In certain embodiments, the invention discloses a method of reducing an individual's likelihood of developing cancer by administering PP2A to the individual, alone or in combination with other phosphatases. In some embodiments, the individual has been determined to have abnormally low PP2A activity, alone or in combination with other phosphatases. In certain embodiments, the PP2A administered is recombinant PP2A. In certain embodiments PP2A is administered via viral vectors. In some embodiments, the viral vector is an adenovirus. One of skill in the art would readily appreciate that any number of other viruses with substantially similar capacities for expression could be used. In yet other embodiments, a substitute agent for PP2A with substantially similar physical properties and/or physiological effect with respect to the ability of PP2A to reduce an individual's likelihood of developing cancer is administered to the individual by one or more methods described herein. In certain embodiments, the individual is a mammal. In certain embodiments, the individual is a human.

In some embodiments, the invention teaches a method of treating an individual after determining the individual has a higher than average risk of developing an infection resulting from abnormally low PP2A activity, including: obtaining a biological sample from the individual; measuring a level of protein phosphatase 2A (PP2A) activity in the biological sample; determining the individual's susceptibility to the infection based upon the level of PP2A activity, wherein a higher than average risk of developing the infection is determined if abnormally low PP2A activity is measured, and an average risk of developing the infection is determined if normal PP2A activity is measured; and treating the individual with a prophylactic dose of a type of medication selected from the group consisting of: antiviral, antifungal, antibacterial, and combinations thereof if it is determined the individual has a higher than average risk of developing the infection resulting from abnormally low PP2A activity.

In certain embodiments, the invention teaches a method of monitoring an individual after determining the individual has a higher than average risk of developing cancer resulting from abnormally low PP2A activity, including: obtaining a biological sample from the individual; measuring a level of protein phosphatase 2A (PP2A) activity in the biological sample; determining the individual's susceptibility to cancer based upon the level of PP2A activity measured, wherein a higher than average risk of developing cancer is determined if abnormally low PP2A activity is measured, and an average risk of developing cancer is determined if normal PP2A activity is measured; and testing the individual for one or more forms of cancer more frequently than an individual having an average risk of developing cancer if it is determined the individual has a higher than average risk of developing cancer resulting from abnormally low PP2A activity.

In certain embodiments, the invention teaches a method of treating an individual after determining the individual has a higher than average risk of developing cancer of the digestive tract resulting from abnormally low PP2A activity, including: obtaining a biological sample from the individual; measuring a level of protein phosphatase 2A (PP2A) activity in the biological sample; determining the individual's susceptibility to cancer of the digestive tract based upon the level of PP2A activity, wherein a higher than average risk of developing cancer of the digestive tract is determined if abnormally low PP2A activity is measured, and an average risk of developing cancer of the digestive tract is determined if normal PP2A activity is measured; and treating the individual with antibiotics if it is determined the individual has a higher than average risk of developing cancer of the digestive tract resulting from abnormally low PP2A activity. In some embodiments, the individual has been diagnosed with inflammatory bowel disease.

In certain embodiments, the gram-negative and gram-positive bacteria associated with one or more types of infection disclosed herein, include: Enterobacter, Staphylococcal, and Streptococcal species. In certain embodiments, specific organisms include but are not limited to Escherichia coli, Salmonella, Shigella, Pseudomonas, Moraxella, Helicobacter, Stenotrophomonas, Bdellovibrio, Legionella, Neisseria gonorrhoeae, Neisseria meningitidis, Moraxella catarrhalis, Hemophilus influenzae, Klebsiella pneumoniae, Legionella pneumophila, Pseudomonas aeruginosa, Proteus mirabilis, Enterobacter cloacae, Serratia marcescens, Helicobacter pylori, Salmonella enteritidis, Salmonella typhi, Acinetobacter species, Klebsiella species, Burkholderia, Staphylococcus aureus (including Methicillin resistant staphylococcus aureus), Streptococcus pneumonia, Group A streptococcus, Group B streptococcus, Coagulase negative staphylococcus, Listeria monocytogeneses, and combinations thereof. In certain embodiments, the virus or viruses associated with viral infections include but are not limited to Influenza, parainfluenza, respiratory syncytial virus, adenovirus, rhinovirus, measles, metapneumovirus, SARS and combinations thereof. In certain embodiments the yeast associated with infections may include but are not limited to species of Candida. In certain embodiments, the mold associated with infection may include but are not limited to species of Aspergillus and mucormycosis.

In various embodiments, the present invention provides pharmaceutical compositions including a pharmaceutically acceptable excipient along with a prophylactically or therapeutically effective amount of PP2A, recombinant PP2A, or a substitute agent with substantially similar physical properties and/or physiological effect as those attributed to PP2A as described herein, alone or in combination with a prophylactically or therapeutically effective amount of other phosphatases. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, viral vector or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its prophylactic or therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, an elixir, an emulsion or an aqueous or non-aqueous suspension. Such a liquid formulation may be administered directly p.o. or filled into a soft gelatin capsule.

The pharmaceutical compositions according to the invention may be delivered in a prophylactically or therapeutically effective amount. The precise prophylactically or therapeutically effective amount is that amount of the composition that will yield the most effective results in terms of efficacy of treatment in a given subject. This amount will vary depending upon a variety of factors, including but not limited to the characteristics of the compound and/or viral vector (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a prophylactically or therapeutically effective amount through routine experimentation, for instance, by monitoring a subject's response to administration of a compound/vector and adjusting the dosage accordingly. For additional guidance, see Remington: The Science and Practice of Pharmacy (Gennaro ed. 20th edition, Williams & Wilkins PA, USA) (2000).

Typical dosages can be in the ranges recommended by the manufacturer where known prophylactic or therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the prophylactic or therapeutic method based, for example, on in vitro responsiveness or the responses observed in the appropriate animal models.

The present invention is also directed to a kit with an intended function selected from the group consisting of: preventing, lessening, diagnosing, prognosing, and treating immune suppression, infection, sepsis and the associated risk of cancer development. The kit can be configured in numerous ways to be useful for practicing any of the inventive methods disclosed herein, including: preventing, lessening, diagnosing, prognosing, and treating infection, sepsis, immune suppression, and the associated risk of cancer development secondary to infection. The kit is an assemblage of materials or components, including at least one of the inventive compositions. Thus, in some embodiments the kit contains a composition including PP2A, with or without other phosphatases, as described above. In other embodiments, the kit contains a composition and/or components useful for measuring the level of PP2A activity directly and/or indirectly, and/or other phosphatases in a subject, as described herein.

The exact nature of the components configured in the inventive kit depends upon its intended purpose. For example, some embodiments are configured for the purpose of diagnosing or treating infection or determining the risk thereof. Other embodiments are configured for the purpose of diagnosing or treating immune suppression or determining the risk thereof. Other embodiments are configured for the purpose of treating cancerous conditions or determining the risk thereof. Some embodiments are configured for determining the aforementioned risks by proving a means for measuring PP2A activity directly or indirectly, alone or together with the activity of other phosphatases. In the event the kit is configured for treating one or more of the conditions described above, it includes a quantity of PP2A, or a functionally equivalent substitute with the same physiological effect. In one embodiment, the kit is configured particularly for use with mammalian subjects. In another embodiment, the kit is configured particularly for use with human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome, such as to treat an infection, prevent an infection, or to diagnose susceptibility to an infection, sepsis, or a cancerous condition secondary to infection. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in treating, detecting or preventing infection, or cancer secondary thereto. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing PP2A and/or other phosphatases and/or functional equivalents thereof, or a substance useful in detecting the activity of PP2A directly and/or indirectly in a subject. The package may also contain materials to measure the level of other phosphatases. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described.

The following examples are for illustrative purposes only and are not intended to limit the scope of the disclosure or its various embodiments in any way.

### EXAMPLES

### Example 1

### Materials

Ultrapure LPS from E. coli 0111:B4 was purchased from InvivoGen (San Diego, Calif.). PP2A Immunoprecipitation Phosphatase Assay Kit was purchased from Upstate (Lake Placid, N.Y.).

### Example 2

### Mice

Six-week-old C57BL/6 mice were purchased from the Jackson Laboratory (Bar Harbor, Me.). Mice were maintained under specific pathogen-free conditions in the Animal Care Facility at Cedars-Sinai Medical Center. The mice used in all experiments were handled according to the guidelines and approved protocols of the Cedars-Sinai Medical Center Animal Care and Use Committees (IACUC 2700).

### Example 3

### Cell Culture and Induction of Tolerance

The human acute monocytic leukemia cell line, THP-1, was obtained from the American Type Culture Collection and maintained in RPMI 1640 medium (Invitrogen Life Technologies) supplemented with 10 U/ml penicillin G, 10 μg/ml streptomycin, 2 mM L-glutamine, and 10% FBS at 37° C. and 5% CO₂ in a humidified incubator. LPS-tolerant THP-1 cells were made and are characterized in detail as described previously (35). Cells were rendered LPS tolerant by preincubation over 16 h with LPS (10 ng/ml LPS from Escherichia coli 0111:B4). Low passage number and log-phase cells were used for all experiments.

### Example 4

### Isolation of peritoneal macrophages

Resident peritoneal macrophages were obtained from C57BL/6 mice by peritoneal lavage. Cells were washed with PBS and seeded in 96-well plates. After 2 h cells were washed again and adherent cells were used for experiments. Cells were cultured in RPMI supplemented with 10% FBS and preincubated with LPS (10 ng/ml) overnight. On the next day, cells were washed with PBS and stimulated with 100 ng/ml LPS or medium for 5 h. Supernatants were harvested and stored at −80° C.

### Example 5

### Cytokine Measurements

Human TNF-α, mouse TNF-α, and mouse IL-6 were quantified by ELISA (eBioscience) in supernatants or serum samples.

### Example 6

### Phosphatase Activity Assay

The activity of PP2A in cell lysates was determined as recommended by the manufacturer (Upstate). In brief, cells were incubated for the indicated time-points, placed on ice and washed with ice-cold PBS/orthovanadate (1 mM). Cells were lysed in phosphate extraction buffer (20 mM imidazole, 2 mM EDTA, 2 mM EGTA, pH 7.0 containing protease inhibitor cocktail) for 20 min. on ice. Cellular lysates were cleared by centrifugation (20 min, 4° C., 2000×g). Protein concentrations were determined by Bradford assay and equal amounts of protein were incubated with anti-PP2A and Protein A agarose beads for 2 h at 4° C. with constant rocking Phosphopeptide substrate was added (final concentration 750 μM) and incubated for 10 min. at 30° C. in a shaking incubator. Samples were centrifuged and added to a microtiter plate. 10 to 15 min. after the addition of Malachite Green Phosphate Detection Solution the plate was read at 650 nm in a microplate reader. The PP2A activity was calculated using a phosphate standard curve.

### Example 7

### In Vivo Induction of Tolerance

LPS tolerance was induced in mice as described previously (36). In brief, mice were i.p. injected with either 25 μg LPS or PBS. 20 h after the first injection, mice were i.p. injected with either 25 μg LPS or PBS. Serum was collected from mice 1.5, 6, and 24 h after the 2^{nd} LPS injection. Peritoneal macrophages were collected 24 h after the 2^{nd} LPS injection and assessed for PP2A activity.

### Example 8

### Statistics

Data shown are the mean±SD of three or more independent experiments. Student's 2-tailed t-test was used to compare groups. Unless otherwise noted, p<0.05 was considered significant.

### Example 9

### PP2A Activity is Reduced in LPS-Tolerized THP-1 Cells

Serine-threonine phosphatases play an important role in LPS-induced signal transduction. Treatment of macrophages with the PP1/PP2A inhibitor calyculin A inhibits LPS-induced macrophage responses (32).

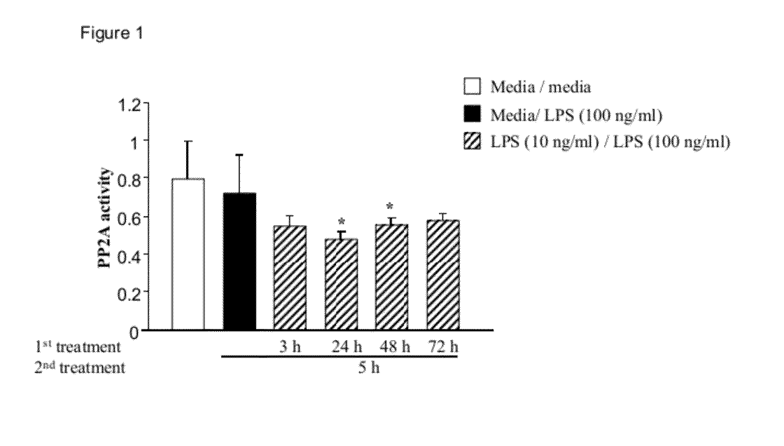

The inventors hypothesized that serine-threonine phosphatases may have a role in LPS-induced macrophage tolerance. In order to test this hypothesis, the inventors used the THP-1 human macrophage cell line and treated with either 10 ng/ml LPS or media for various durations followed by a second stimulation with 100 ng/ml LPS for 5 h. PP2A activity was determined 5 h after 2^{nd} LPS treatment. The inventors observed that repeated LPS stimulation of THP-1 cells led to significantly reduced PP2A activity compared to THP-1 cells that only received the 2^{nd} LPS treatment (FIG. 1). The reduction in PP2A activity was most prominent after 24 and 48 h of primary LPS exposure (FIG. 1). The inventors observed the greatest reduction in PP2A activity in THP-1 cells that were pre-treated with LPS for 24 h.

### Example 10

### PP2A Activity is Reduced in In Vivo Tolerized Peritoneal Macrophages

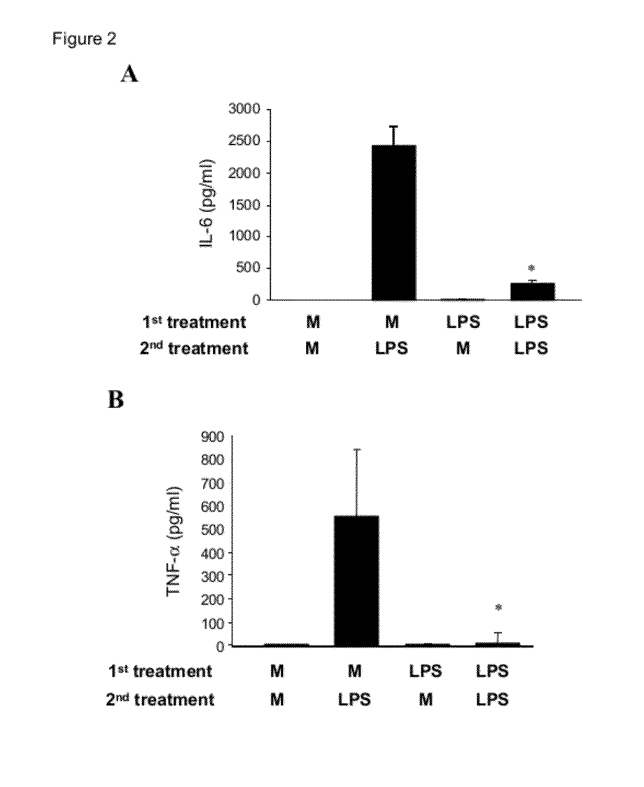

In order to confirm the in vitro findings in an in vivo animal model the inventors first determined whether murine peritoneal macrophages could be tolerized under the same conditions. Peritoneal macrophages from C57BL/6 mice were isolated by peritoneal lavage and stimulated with 10 ng/ml LPS overnight. After the second LPS stimulation (100 ng/ml for 5 h) IL-6 and TNF-α release into the supernatants were determined by ELISA (FIG. 2). The inventors observed that repeated exposure to LPS led to the induction of tolerance in murine peritoneal macrophages. IL-6 (FIG. 2A) as well as TNF-α (FIG. 2B) release from LPS/LPS stimulated macrophages was significantly reduced compared to medium/LPS treated macrophages.

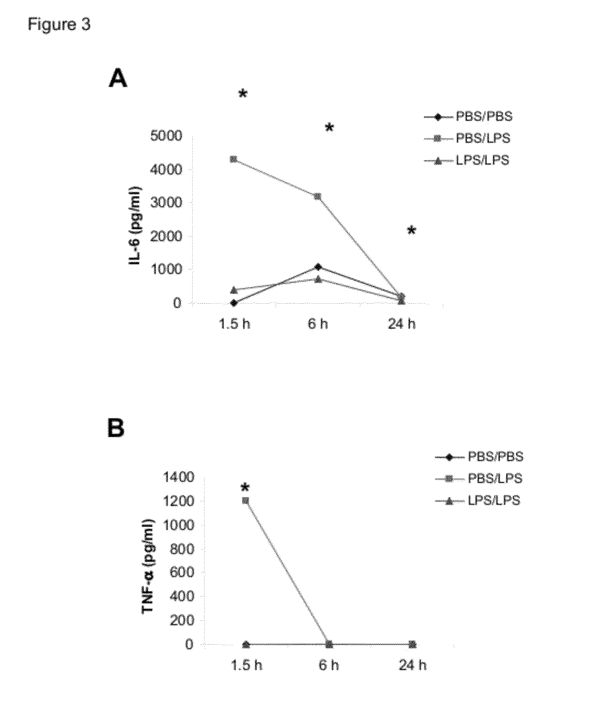

Next, the inventors tolerized C57BL/6 mice in vivo by repeated injections of LPS. LPS (25 μg/mouse) was injected i.p. followed by a 2^{nd} injection of LPS (25 μg/mouse) 20 h later. Serum was collected at 1.5, 6, and 24 h later, and TNF-α and IL-6 levels were measured to confirm induction of tolerance (FIG. 3). Mice injected with LPS/LPS had significantly reduced IL-6 serum concentrations compared to mice injected with medium/LPS at 1.5, 6, and 24 h after the 2^{nd} LPS injection (FIG. 3A) (at 1.5 h: 376±200 vs. 4286±1645 pg/ml for LPS/LPS and PBS/LPS, respectively, p<0.05; at 6 h: 722±398 vs. 3163±741 pg/ml for LPS/LPS and PBS/LPS, respectively, p<0.05; at 24 h: 76±15 vs. 168±43 pg/ml for LPS/LPS and PBS/LPS, respectively, p<0.05). inventors also observed significantly reduced TNF-α serum concentrations in mice injected with LPS/LPS compared to mice injected with medium/LPS at 1.5 h after the 2^{nd} LPS injection (FIG. 3B) (0 vs. 1195±578 pg/ml for LPS/LPS and PBS/LPS, respectively, p<0.05). In non-tolerized mice serum concentrations of TNF-α declined to non-detectable at 6 h after the 2^{nd} LPS injection.

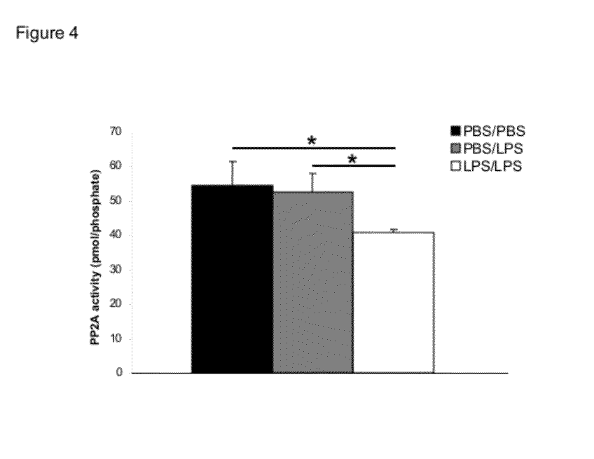

Next, inventors determined the effect of in vivo LPS tolerization of C57BL/6 mouse on PP2A activity. Peritoneal macrophages of tolerized and non-tolerized mice were harvested 24 h after the 2^{nd} injection of LPS and PP2A activity was determined. As shown in FIG. 4 PP2A activity was significantly reduced in tolerized mice (LPS/LPS) compared to mice that received only the 2^{nd} LPS injection (PBS/LPS) or mice that were injected with PBS alone (PBS/PBS). These data suggest that changes in the macrophage PP2A activity are associated with the development of LPS tolerance in mice.

### Example 11

### Recombinant PP2A Restores LPS Responsiveness in LPS-Tolerized THP-1 Cells

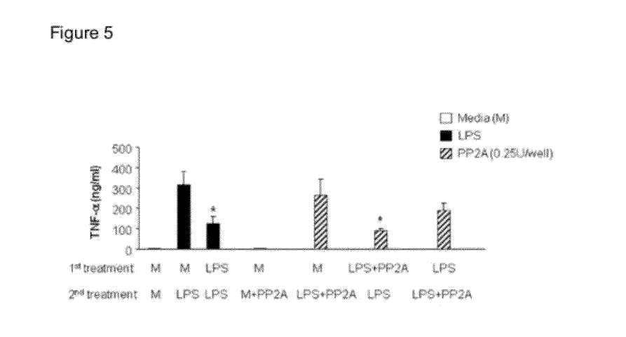

The inventors also determined that exogenous recombinant PP2A is able to restore LPS responsiveness in THP-1 cells. THP-1 cells were tolerized to LPS by overnight stimulation with low-dose LPS (10 ng/ml). The next day, cells were stimulated with 100 ng/ml LPS for 5 h. TNF-α release as measured by ELISA confirmed that THP-1 cells lost LPS responsiveness after overnight exposure to LPS (FIG. 5). To elucidate the mechanism of LPS tolerance THP-1 cells were pre-incubated with recombinant PP2A prior to the 2nd exposure with LPS. Pre-incubation with PP2A restored LPS responsiveness in THP-1 cells. Interestingly, incubation of THP-1 cells with PP2A during the first exposure with LPS did not restore LPS responsiveness

### Example 12

### Discussion

As disclosed herein, the inventors show that the serine/threonine protein phosphatase 2 A (PP2A) activity is suppressed in in vitro tolerized human macrophage cell line as well as in peritoneal macrophages isolated from in vivo LPS-tolerized mice.

In tolerized THP-1 cells, suppression of PP2A activity was time-dependent. The inventors observed that 24 and 48 h of pretreatment with LPS led to the greatest reduction in PP2A activity. Pretreatment with LPS for longer than 48 h (i.e. 72 h) resulted in only slightly reduced PP2A activity after the 2^{nd} LPS stimulation. This transient down-regulation of PP2A activity correlates with previous observations on the duration of immune suppression in LPS tolerized macrophages. Zuckerman et al. have shown that LPS responsiveness was restored in macrophages after 48 h of LPS stimulation (8).

Phosphatases play a crucial role in the tight regulation of LPS-induced signaling events. The transient phosphorylation of signaling molecules that leads to the activation of transcription factors such as NF-κB guarantees protection against over-stimulation of the signaling pathways that induce inflammation. Phosphatases have been implemented to play an important role in LPS tolerance (37). Expression of the SH2-containing inositol phosphatase 1 (SHIP-1) for instance is required for the development of an LPS-refractory state in macrophages and mice (38) as macrophages from SHIP-deficient mice do not develop endotoxin tolerance. Serine/threonine phosphatases play an important role in LPS-induced signal transduction. Treatment of macrophages with the PP1/PP2A inhibitor calyculin A inhibits LPS-induced macrophage responses (32).

The serine/threonine phosphatase PP2A has been shown to inactivate PKC by dephosphorylation (39,40) and regulate the MAPK cascade (41). Both pathways have been implicated in endotoxin tolerance (12,42). Ropert et al. have shown that treatment with okadaic acid (OA), an inhibitor of serine/threonine phosphatases PP1 and PP2A 1 h before the first LPS stimulation or 16 h before the second LPS stimulation reversed LPS tolerance in murine peritoneal macrophages (43). OA treatment restored and prolonged the phosphorylation of the ERK-1/ERK-2, JNK/SAPK and p38/SAPK-2 in response to second LPS stimulation in macrophages (43). Interestingly OA treatment led to inhibition of p38 phosphorylation in response to primary LPS stimulation of macrophages (43).

Protein kinase R(PKR) is a serine-threonine kinase that is activated upon LPS stimulation of TLR4 (44). PKR phosphorylates the B56a regulatory subunit of PP2A to increase the activity of trimeric PP2A holoenzyme (45). Recently Vogel et al. have shown that PKR activity is inhibited in LPS-tolerant macrophages (29). These data support the inventors' observations that PP2A activity is suppressed in LPS-tolerized macrophages.

In conclusion the inventors' study demonstrates that in vivo, peritoneal macrophages isolated from tolerized mice had reduced PP2A activity supporting the inventors' in vitro findings of reduced PP2A activity in THP-1 cells.

Finally, repeated infections have been thought to be associated with development of cancers (46), while inhibition of PP2A activity has been suggested to play a role in cancer development (47).

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Wittmann, M., Larsson, V. A., Schmidt, P., Begemann, G., Kapp, A., and Werfel, T. (1999) Blood 94, 1717-1726
2. Wysocka, M., Robertson, S., Riemann, H., Caamano, J., Hunter, C., Mackiewicz, A., Montaner, L. J., Trinchieri, G., and Karp, C. L. (2001) J Immunol 166, 7504-7513
3. Ziegler-Heitbrock, H. W. (1995) J Inflamm 45, 13-26
4. Beeson, P. B. (1947) J Exp Med 86, 29-38
5. Haas, J. G., Baeuerle, P. A., Riethmuller, G., and Ziegler-Heitbrock, H. W. (1990) Proc Natl Acad Sci USA 87, 9563-9567
6. Virca, G. D., Kim, S. Y., Glaser, K. B., and Ulevitch, R. J. (1989) J Biol Chem 264, 21951-21956
7. Ziegler-Heitbrock, H. W., Blumenstein, M., Kafferlein, E., Kieper, D., Petersmann, I., Endres, S., Flegel, W. A., Northoff, H., Riethmuller, G., and Haas, J. G. (1992) Immunology 75, 264-268
8. Zuckerman, S. H., Evans, G. F., Snyder, Y. M., and Roeder, W. D. (1989) J Immunol 143, 1223-1227
9. Granowitz, E. V., Porat, R., Mier, J. W., Orencole, S. F., Kaplanski, G., Lynch, E. A., Ye, K., Vannier, E., Wolff, S. M., and Dinarello, C. A. (1993) J Immunol 151, 1637-1645
10. Cavaillon, J. M., and Adib-Conquy, M. (2006) Crit. Care 10, 233
11. Munoz, C., Carlet, J., Fitting, C., Misset, B., Bleriot, J. P., and Cavaillon, J. M. (1991) J Clin Invest 88, 1747-1754
12. Biswas, S. K., and Lopez-Collazo, E. (2009)Trends Immunol 30, 475-487
13. Lehner, M. D., Morath, S., Michelsen, K. S., Schumann, R. R., and Hartung, T. (2001) J Immunol 166, 5161-5167
14. Medvedev, A. E., Kopydlowski, K. M., and Vogel, S, N. (2000) J Immunol 164, 5564-5574
15. Chang, L., and Karin, M. (2001) Nature 410, 37-40
16. Cohen, P. (1997) Trends Cell Biol 7, 353-361
17. Nomura, F., Akashi, S., Sakao, Y., Sato, S., Kawai, T., Matsumoto, M., Nakanishi, K., Kimoto, M., Miyake, K., Takeda, K., and Akira, S. (2000) J Immunol 164, 3476-3479
18. Adib-Conquy, M., and Cavaillon, J. M. (2002) J Biol Chem 277, 27927-27934
19. Jacinto, R., Hartung, T., McCall, C., and Li, L. (2002) J Immunol 168, 6136-6141
20. Li, L., Cousart, S., Hu, J., and McCall, C. E. (2000) J Biol Chem 275, 23340-23345
21. Li, L., Jacinto, R., Yoza, B., and McCall, C. E. (2003) J Endotoxin Res 9, 39-44
22. Ogawa, H., Rafiee, P., Heidemann, J., Fisher, P. J., Johnson, N. A., Otterson, M. F., Kalyanaraman, B., Pritchard, K. A., Jr., and Binion, D. G. (2003) J Immunol 170, 5956-5964
23. Medvedev, A. E., Lentschat, A., Wahl, L. M., Golenbock, D. T., and Vogel, S, N. (2002) J Immunol 169, 5209-5216
24. Escoll, P., del Fresno, C., Garcia, L., Valles, G., Lendinez, M. J., Arnalich, F., and Lopez-Collazo, E. (2003) Biochem Biophys Res Commun 311, 465-472
25. Kobayashi, K., Hernandez, L. D., Galan, J. E., Janeway, C. A., Jr., Medzhitov, R., and Flavell, R. A. (2002) Cell 110, 191-202
26. Liew, F. Y., Xu, D., Brint, E. K., and O'Neill, L. A. (2005) Nat Rev Immunol 5, 446-458
27. Piao, W., Song, C., Chen, H., Diaz, M. A., Wahl, L. M., Fitzgerald, K. A., Li, L., and Medvedev, A. E. (2009) J Leukoc Biol 86, 863-875
28. Rauh, M. J., Ho, V., Pereira, C., Sham, A., Sly, L. M., Lam, V., Huxham, L., Minchinton, A. I., Mui, A., and Krystal, G. (2005) Immunity 23, 361-374
29. Perkins, D. J., Qureshi, N., and Vogel, S, N. (2010) MBio 1
30. Junttila, M. R., Li, S. P., and Westermarck, J. (2008) Faseb J22, 954-965
31. Shi, Y. (2009) Cell 139, 468-484
32. Barber, S. A., Perera, P. Y., McNally, R., and Vogel, S, N. (1995) J Immunol 155, 1404-1410
33. Kray, A. E., Carter, R. S., Pennington, K. N., Gomez, R. J., Sanders, L. E., Llanes, J. M., Khan, W. N., Ballard, D. W., and Wadzinski, B. E. (2005) J Biol Chem 280, 35974-35982
34. Li, S., Wang, L., Berman, M. A., Zhang, Y., and Dorf, M. E. (2006) Mol Cell 24, 497-509
35. LaRue, K. E., and McCall, C. E. (1994) J Exp Med 180, 2269-2275
36. Madonna, G. S., and Vogel, S, N. (1985) J Immunol 135, 3763-3771
37. Rauh, M. J., Sly, L. M., Kalesnikoff, J., Hughes, M. R., Cao, L. P., Lam, V., and Krystal, G. (2004) Biochem Soc Trans 32, 785-788
38. Sly, L. M., Rauh, M. J., Kalesnikoff, J., Song, C. H., and Krystal, G. (2004) Immunity 21, 227-239
39. Ricciarelli, R., and Azzi, A. (1998) Arch Biochem Biophys 355, 197-200
40. Srivastava, J., Goris, J., Dilworth, S. M., and Parker, P. J. (2002) FEBS Lett 516, 265-269
41. Silverstein, A. M., Barrow, C. A., Davis, A. J., and Mumby, M. C. (2002) Proc Natl Acad Sci USA 99, 4221-4226
42. Cuschieri, J., Billigren, J., and Maier, R. V. (2006) J Leukoc Biol 80, 1289-1297
43. Ropert, C., Closel, M., Chaves, A. C., and Gazzinelli, R. T. (2003) J Immunol 171, 1456-1465
44. Horng, T., Barton, G. M., and Medzhitov, R. (2001) Nat Immunol 2, 835-841
45. Xu, Z., and Williams, B. R. (2000) Mol Cell Biol 20, 5285-5299
46. Rakoff-Nahoum S, Medzhitov R. 2009. Toll-like receptors and cancer. Nat Rev Cancer. 9:57-63.
47. Westermarck J, Hahn W C. Multiple pathways regulated by the tumor suppressor PP2A in transformation. Trends Mol. Med. 2008; 14:152-60.

What is claimed is:

1. A method of determining an individual's susceptibility to an infection, comprising:
obtaining a biological sample from the individual;
measuring a level of protein phosphatase 2A (PP2A) activity in the biological sample; and
determining the individual's susceptibility to the infection based upon the level of PP2A activity, wherein a higher than average risk of developing an infection is determined if abnormally low PP2A activity is measured, and an average risk of developing an infection is determined if normal PP2A activity is measured.

2. The method of claim 1, wherein the level of PP2A activity is measured indirectly by measuring the level of phosphorylation of its upstream and/or downstream interacting/target molecules in the biological sample.

3. The method of claim 1, wherein the individual is immune suppressed.

4. The method of claim 1, wherein the individual has sepsis.

5. The method of claim 1, wherein the infection is a superinfection.

6. The method of claim 1, wherein the infection is selected from the group consisting of: bacterial, fungal, viral, and combinations thereof.

7. The method of claim 1, wherein the individual has a history of infection.

8. The method of claim 1, wherein the infection is a gram-negative and/or gram-positive bacterial infection.

9. A method of reducing a likelihood of an individual developing an infection, comprising:
providing a prophylactic dose of protein phosphatase 2A (PP2A); and
administering the dose to the individual.

10. The method of claim 9, wherein the individual is immune suppressed.

11. The method of claim 9, wherein the infection is a superinfection.

12. The method of claim 9, wherein the infection is selected from the group consisting of:
bacterial, fungal, viral, and combinations thereof.

13. The method of claim 9, wherein the infection is a gram-negative and/or gram-positive bacterial infection.

14. The method of claim 9, wherein the individual has sepsis, and the infection is secondary infection.

15. A method of treating an infection in an individual, comprising:
providing a therapeutic dose of protein phosphatase 2A (PP2A); and
administering the dose to the individual.

16. The method of claim 15, wherein the individual is immune suppressed.

17. The method of claim 15, wherein the infection is a superinfection.

18. The method of claim 15, wherein the infection is selected from the group consisting of: bacterial, fungal, viral, and combinations thereof.

19. The method of claim 15, wherein the infection is a gram-negative and/or gram-positive infection.

20. A kit for determining an individual's susceptibility to infection, comprising:
a means for measuring a level of protein phosphatase 2A (PP2A) activity in a biological sample of the individual, and
instructions for use thereof to determine an individual's susceptibility to infection.

21. The kit of claim 20, wherein the level of PP2A activity is measured indirectly by measuring the level of phosphorylation of its upstream and/or downstream interacting/target molecules in the biological sample.

22. The kit of claim 20, wherein the infection is selected from the group consisting of: viral, bacterial, fungal, and combinations thereof.

23. A kit for treating an infection in an individual, comprising:
a quantity of protein phosphatase 2A (PP2A), and
instructions for use thereof to treat an infection in an individual.

24. The kit of claim 23, wherein the infection is selected from the group consisting of: viral, bacterial, fungal, and combinations thereof.

25. A kit for reducing the likelihood of an individual contracting an infection, comprising:
a quantity of protein phosphatase 2A (PP2A), and
instructions for use thereof for reducing the likelihood of an individual contracting an infection.

26. The kit of claim 25, wherein the infection is selected from the group consisting of: viral, bacterial, fungal, and combinations thereof.

27. A method of determining an individual's susceptibility to cancer, comprising:
obtaining a biological sample from the individual;
measuring a level of protein phosphatase 2A (PP2A) activity in the biological sample; and
determining the individual's susceptibility to cancer based upon the level of PP2A activity, wherein a higher than average risk of developing cancer is determined if abnormally low PP2A activity is measured, and an average risk of developing cancer is determined if normal PP2A activity is measured.

28. The method of 27, wherein the level of PP2A activity is measured indirectly by measuring the level of phosphorylation of its upstream and/or downstream interacting/target molecules in the biological sample.

29. The method of claim 27, wherein the cancer is the result of repeated infections and/or reduced PP2A activity.

30. A kit for determining an individual's susceptibility to cancer, comprising:
a means for measuring a level of protein phosphatase 2A (PP2A) activity in a biological sample of the individual, and
instructions for use thereof to determine an individual's susceptibility to cancer.

31. The kit of claim 30, wherein the level of PP2A activity is measured indirectly by measuring the level of phosphorylation of its upstream and/or downstream interacting/target molecules in the biological sample.

32. The kit of claim 30, wherein the cancer is the result of repeated infections and/or reduced PP2A activity.

33. A method of treating an individual after determining the individual has a higher than average risk of developing an infection resulting from abnormally low PP2A activity, comprising:
obtaining a biological sample from the individual;
measuring a level of protein phosphatase 2A (PP2A) activity in the biological sample;
determining the individual's susceptibility to the infection based upon the level of PP2A activity, wherein a higher than average risk of developing the infection is determined if abnormally low PP2A activity is measured, and an average risk of developing the infection is determined if normal PP2A activity is measured; and
treating the individual with a prophylactic dose of a type of medication selected from the group consisting of: antiviral, antifungal, antibacterial, and combinations thereof if it is determined the individual has a higher than average risk of developing the infection resulting from abnormally low PP2A activity.

34. A method of monitoring an individual after determining the individual has a higher than average risk of developing cancer resulting from abnormally low PP2A activity, comprising:
obtaining a biological sample from the individual;
measuring a level of protein phosphatase 2A (PP2A) activity in the biological sample;
determining the individual's susceptibility to cancer based upon the level of PP2A activity measured, wherein a higher than average risk of developing cancer is determined if abnormally low PP2A activity is measured, and an average risk of developing cancer is determined if normal PP2A activity is measured; and
testing the individual for one or more forms of cancer more frequently than an individual having an average risk of developing cancer if it is determined the individual has a higher than average risk of developing cancer resulting from abnormally low PP2A activity.

35. A method of treating an individual after determining the individual has a higher than average risk of developing cancer of the digestive tract resulting from abnormally low PP2A activity, comprising:
obtaining a biological sample from the individual;
measuring a level of protein phosphatase 2A (PP2A) activity in the biological sample;
determining the individual's susceptibility to cancer of the digestive tract based upon the level of PP2A activity, wherein a higher than average risk of developing cancer of the digestive tract is determined if abnormally low PP2A activity is measured, and an average risk of developing cancer of the digestive tract is determined if normal PP2A activity is measured; and
treating the individual with antibiotics if it is determined the individual has a higher than average risk of developing cancer of the digestive tract resulting from abnormally low PP2A activity.

36. The method of claim 35, wherein the individual has been diagnosed with inflammatory bowel disease.
